SEQUENCE LISTING (11 sequences)

SEQ ID NO: 1 - RNA, 5292 nt, Homo sapiens
  Homo sapiens CD47 molecule (CD47), transcript variant 1, mRNA, Accession no. NM_001777, version no. NM_001777.4
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 2 - RNA, 5234 nt, Homo sapiens
  Homo sapiens CD47 molecule (CD47), transcript variant 2, mRNA, accession no. NM_198793, version NM_198793.3
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 3 - RNA, 2589 nt, Mus musculus
  Mus musculus CD47 antigen (Rh-related antigen, integrin-associated signal transducer) (Cd47), transcript variant 1, mRNA, accession no. NM_001368415 XM_006521811, version no. NM_001368415.1
agcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaggaaaaaaaaaaaaagctatagttgtaacatatgtattccagacctctggtttaaaggcaaaagaaaaaaaatctacagtgtttcttctcatgttttctgatcggaggcatgacaaagcaagactgaaatctgaactgtgtctcctgcatggcaacacgtgtctccgtcaggccctcgcaaggcccggggagggggttctacgcctcttgtctctttgttgcatgctgaacactcatcgccttcctactgtatcctgcctcctgcagcctccctcttcctcctcctcttcctcttcctcctcttcctcctcctcctcctcttcctccaagtttgaaaggtcaaacaaaactaccacattccctacccagttagaagaaaaccaccgtcctgacagttgtgatcgcatggagtacttttagattattagcacctgtttttacctcgtttgtgggcgtgtttgtatgtgcacatgtatgaagtcggcacatgcaccttctgtatgggcagaggcgtggcatctacagaagagcagatgccaactttgtgcttttagtgaatacattaaaaaaaaaaaaccaacggtccttattgagtggaattctatttgatgcaaatatttgagctctttaagactttaaaactagataatgtgccaagcttttaggactgctcaccagtgccctctgaagaaacaccagtactttttcctgtttgtgtaataaaggcatatttgtatttg

SEQ ID NO: 4 - RNA, 2710 nt, Mus musculus
  Mus musculus CD47 antigen (Rh-related antigen, integrin-associated signal transducer) (Cd47), transcript variant 2, mRNA, Accession no. NM_001368416 XM_006521806, version no. NM_001368416.1
agcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaaagctgtagaggaaccccttaacgcatttaaagagtcaaaaggaatgatgaatgacgaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaggaaaaaaaaaaaaagctatagttgtaacatatgtattccagacctctggtttaaaggcaaaagaaaaaaaatctacagtgtttcttctcatgttttctgatcggaggcatgacaaagcaagactgaaatctgaactgtgtctcctgcatggcaacacgtgtctccgtcaggccctcgcaaggcccggggagggggttctacgcctcttgtctctttgttgcatgctgaacactcatcgccttcctactgtatcctgcctcctgcagcctccctcttcctcctcctcttcctcttcctcctcttcctcctcctcctcctcttcctccaagtttgaaaggtcaaacaaaactaccacattccctacccagttagaagaaaaccaccgtcctgacagttgtgatcgcatggagtacttttagattattagcacctgtttttacctcgtttgtgggcgtgtttgtatgtgcacatgtatgaagtcggcacatgcaccttctgtatgggcagaggcgtggcatctacagaagagcagatgccaactttgtgcttttagtgaatacattaaaaaaaaaaaaccaacggtccttattgagtggaattctatttgatgcaaatatttgagctctttaagactttaaaactagataatgtgccaagcttttaggactgctcaccagtgccctctgaagaaacaccagtactttttcctgtttgtgtaataaaggcatatttgtatttg

SEQ ID NO: 5 - RNA, 21 nt, Artificial Sequence
  siRNA specific for Homo sapien CD47
ccucuuugaagauggauaatt

SEQ ID NO: 6 - RNA, 29 nt, Artificial Sequence
  siRNA specific for Homo sapien CD47
aagatggataagagtgatgctcctgtctc

SEQ ID NO: 7 - RNA, 18 nt, Artificial Sequence
  siRNA specific for Mus musculus CD47
gaaugaccucuuucacca

SEQ ID NO: 8 - RNA, 29 nt, Artificial Sequence
  siRNA specific for Mus musculus CD47
aagatggataagagtgatgctcctgtctc

SEQ ID NO: 9 - DNA, 25 bp, Artificial Sequence
  Antisense morpholino oligonucleotide specific for Homo sapien CD47
cgtcacaggcaggacccactgccca

SEQ ID NO: 10 - DNA, 29 bp, Artificial Sequence
  Antisense morpholino oligonucleotide specific for Mus musculus CD47
aagatggataagagtgatgctcctgtctc

SEQ ID NO: 11 - DNA, 25 bp, Artificial Sequence
  Antisense morpholino oligonucleotide specific for Mus musculus CD47 (control)
aacaggcaaactgtgtcacttaccc

(19) United States
(12) Patent Application Publication
(10) Pub. No.: US 2024/0091270 A1
(43) Pub. Date: Mar. 21, 2024

(54) MODULATING INSULIN EXPRESSION AND PRODUCTION BY TARGETING CD47

(71) Applicants: THE WESTMEAD INSTITUTE FOR MEDICAL RESEARCH, Westmead, New South Wales (AU); WESTERN SYDNEY LOCAL HEALTH DISTRICT, Westmead, New South Wales (AU)

(72) Inventors: Kedar Ghimire, Glenfield (AU); Natasha Rogers, Westmead (AU)

(21) Appl. No.: 18/033,760

(22) PCT Filed: Oct. 26, 2021
(86) PCT No.: PCT/AU2021/051241

§ 371 Date: Apr. 25, 2023

(30) Foreign Application Priority Data
Oct. 26, 2020 (AU) 2020903869

(51) Int. Cl. A61K 35/39; A61P 3/10; C07K 16/28; C12N 5/071; C12N 15/113

(52) U.S. Cl. CPC A61K 35/39; A61P 3/10; C07K 16/2803; C12N 5/0676; C12N 15/1138; A61K 2039/505

(57) ABSTRACT

The present invention generally relates to modulating insulin expression and production by targeting CD47. In a particular aspect, the invention relates increasing insulin expression by silencing or blocking CD47. In another aspect, the present invention relates to the delivery of insulin to a subject by pancreatic beta islet cells. Specifically, the present invention relates to the production of modified pancreatic beta islet cells that have increased insulin expression and production resulting from silencing or blocking CD47, which are suitable for beta cell transplantation, and methods of their production.

### TECHNICAL FIELD

The present invention generally relates to compositions and methods for modulating insulin expression and production by targeting CD47. The invention also relates to the treatment of a disease or condition (such as prediabetes or diabetes) treatable by increasing insulin expression in a subject in need by manipulating CD47 levels. In a particular aspect, the invention relates to increasing insulin expression and secretion, and consequently decreasing blood sugar levels, by silencing or blocking CD47. In another aspect, the present invention relates to the delivery of insulin to a subject by pancreatic beta islet cells. Specifically, the present invention relates to modified pancreatic beta islet cells that have increased insulin expression resulting from silencing or blocking CD47, which are suitable for beta cell transplantation, and methods of their production.

### BACKGROUND

Blood sugar regulation is the process by which the levels of blood sugar, primarily glucose, are maintained by the body through tight regulation. This tight regulation is referred to as glucose homeostasis. Insulin, which lowers blood sugar, and glucagon, which raises it, are key hormones which are fundamental to glucose homeostasis.

Insulin is a peptide produced by beta cells of the islets of the pancreas, and specifically regulates the metabolism of carbohydrates, fats and protein by promoting the absorption of glucose from the blood into liver, fat and skeletal muscle cells. Circulating insulin also affects the synthesis of proteins in a wide variety of tissues. Insulin is therefore an anabolic hormone, promoting the conversion of small molecules in the blood into large molecules inside the cells. Low insulin levels in the blood have the opposite effect by promoting widespread catabolism, especially of reserve body fat.

Decreased insulin activity, or a complete loss of activity, results in diabetes mellitus or ‘diabetes’, a condition where blood sugar control is unregulated and characteristically high (hyperglycaemia). In type 1 diabetes, the beta cells are destroyed by an autoimmune reaction so that insulin can no longer be synthesised or secreted into the blood. In type 2 diabetes, the body becomes resistant to the normal effects of insulin and/or gradually loses the capacity to produce enough insulin in the pancreas.

Diabetes is a significant global public health problem, and current global estimates indicate that this condition affects 415 million people and is set to escalate to 642 million by the year 2040.

Unregulated high blood glucose levels can lead to serious life-changing and life-threatening complications. The consequences of hyperglycaemia have been associated with co-morbidities like kidney failure, cardiovascular diseases, various neuropathies, peripheral vascular diseases and stroke. Diabetes is chronic and progressive, and there is no treatment to date to reverse its progression. The general therapeutic approach, in addition to changes in individual's lifestyle and dietary habits, includes the use of insulin supplementation and anti-hyperglycaemia drugs.

A surgical option for treating diabetes (primarily type 1) is islet cell transplantation where beta cells are removed from a donor's pancreas and transferred into a person with diabetes. Typically, donor cells are transplanted into the recipient's liver indirectly via a catheter in the portal vein. The premise of this surgical option is that, once transplanted, the islet cells resume their role of releasing insulin to maintain normal blood sugar levels in response to food, exercise, and other changes in the body.

Beta islet cell transplantation is not without challenges that have hindered its use as a mainstream anti-diabetic therapy to benefit diabetic patients. Rejection of the donor cells presents a significant risk, requiring long term treatment with immunosuppressive therapies. Currently, multiple donor islets are required for a single recipient to achieve insulin independence so improving islet yields from donor pancreases can address the challenges related to severe shortage of donor islets. Additionally, successful transplantation is dependent on preserving islet cell mass, function, and survival in the early transplant period. Accordingly, it is desirable to have methods which improve beta islet cell transplantation outcomes.

Discussion or mention of any piece of prior art in this specification is not to be taken as an admission that the prior art is part of the common general knowledge of the skilled addressee of the specification.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

According to embodiment, the invention provides a method of preparing a transplantable pancreatic islet cell, said method comprising: providing an isolated pancreatic islet cell, and reducing CD47 gene expression or function in said cell to produce a modified pancreatic islet cell.

According to another embodiment, the invention provides a modified pancreatic islet cell in which CD47 gene expression or function is reduced when compared to an unmodified pancreatic islet cell.

According to yet another embodiment, the invention provides a method of treating a disease or condition treatable by increasing insulin expression in a subject in need, said method comprising administering to said subject an effective amount of a CD47-specific silencing molecule, preferably wherein said CD47-specific silencing molecule is a CD47-specific siRNA, blocking antibody or morpholino.

In an alternative embodiment, the invention provides a method of treating diabetes in a subject in need, the method comprising modulating CD47 expression or function in said subject. Preferably, treatment comprises administering to said subject an effective amount of a CD47-specific silencing molecule, further preferably wherein said CD47-specific silencing molecule is a CD47-specific siRNA, blocking antibody or morpholino.

According to yet another embodiment, the invention provides for the use of a modified pancreatic islet cell in the preparation of a medicament for treating a disease or condition treatable by pancreatic islet cell transplantation, wherein said modified pancreatic islet cell has reduced CD47 gene expression or function when compared to an unmodified pancreatic islet beta cell.

According to yet another embodiment, the invention provides for the use of gene silencing in the preparation of a modified pancreatic islet cell for treating a condition treatable by pancreatic islet cell transplantation, wherein said gene silencing reduces CD47 expression or function compared to an unmodified pancreatic islet beta cell.

According to yet another embodiment, the invention provides for the use of a CD47-specific silencing molecule, preferably wherein said CD47-specific silencing molecule is a CD47-specific siRNA, blocking antibody or morpholino, in the preparation of a medicament for treating a disease or condition treatable by increasing insulin expression in a subject in need.

Other embodiments of the invention will be evident from the following detailed description of various aspects of the invention.

### DESCRIPTION OF FIGURES

FIG. 1. Shows the effects of CD47 blockade or CD47 deletion on glucose homeostasis and glucose-stimulated insulin secretion respectively in mice. FIG. 1A shows the effect of CD47 blockade on glucose homeostasis in male wildtype (WT) C57/BL6 mice. The left panel shows a 2-hour treatment of mice with anti-CD47 blocking antibody (MIAP301) (N=10 mice/group) compared to a control antibody (IgG), followed by intraperitoneal injection of glucose bolus, whereas the right panel shows the calculation of the area under curve (AUC) of the glucose measurements, where a Student's t-test was used to calculate p-value (**p≤0.01). FIG. 1B shows the differences in plasma insulin levels between WT and CD47^{-/-} mice at different time points after glucose injection, whereas the right panel shows the calculation of the area under curve (AUC) of the glucose measurements, where a Student's t-test was used to calculate p-value (*p<0.05).

FIG. 2. Shows the effect of reducing CD47 gene expression on insulin gene expression in murine and human-derived pancreatic beta islet cells in vitro. FIG. 2A and FIG. 2B show the effect of reducing CD47 gene expression by siRNA knockdown in glucose-stimulated human and murine pancreatic beta islet cells, respectively relative to vinculin expression. FIG. 2C and FIG. 2D show the effect of reducing CD47 gene expression with a CD47 morpholino in glucose-stimulated human pancreatic beta islet cells and the effect of blocking CD47 with an anti-CD47 antibody (MIAP301) in murine beta islet cells respectively relative to vinculin expression.

FIG. 3. Shows the effect of reducing CD47 on insulin production and secretion in vitro from murine and human pancreatic beta islet cells. FIG. 3A shows changes in glucose-stimulated insulin secretion (GSIS) from mouse pancreatic islet cells, achieved by reducing CD47 levels by siRNA treatment. FIG. 3B shows the difference in insulin protein content in mouse pancreatic islet cells, achieved by manipulating CD47 levels using siRNA. FIG. 3C shows changes in GSIS from human pancreatic islets, achieved by reducing CD47 levels using a CD47 morpholino. FIG. 3D shows the difference in insulin protein content in human pancreatic islets, achieved by reducing CD47 levels using a CD47 morpholino.

FIG. 4. Shows the effect of CD47 depletion on glycemic control in diabetic C57/BL6 mice transplanted with pancreatic beta islet cells. FIG. 4A shows the difference in islet transplantation efficiency and glycemic control achieved in diabetic mice transplanted with equal number (400) non-diabetic WT donor islets or islets from CD47^{-/-} mice. A red horizontal line represents the average normal blood glucose level (BGL) of these mice when non-diabetic. FIG. 4B shows islet transplantation efficiency and glycemic control achieved by reducing CD47 levels therapeutically by pre-treating donor islets with MIAP301 versus a control IgG.

FIG. 5. Shows the effect of CD47 depletion on glycemic control in diabetic C57/BL6 mice transplanted with pancreatic beta islet cells. Specifically, the figure combines the data shown in FIG. 4A and FIG. 4B.

FIG. 6. Shows the impact of CD47 deletion in aged CD47^{-/-} and WT C57/BL6 mice fed a high fat diet. FIG. 6A is a photograph of an 18-month old CD47^{-/-} mouse next to a WT C57/BL6 mouse, both fed a high fat diet (HFD). FIG. 6B shows prevention of HFD-induced weight gain with age by deleting CD47 in mice and the average body weight differences between the two groups of mice at different ages. Ordinary one-way Anova with multiple comparisons was used to calculate p-value, with *p≤0.05. FIG. 6C shows the blood glucose level in 18-month old male CD47^{-/-} and WT C57/BL6 mice after a 6 hour fast (N=10-12 mice/group). Two-way Anova with multiple comparisons was used to calculate p-value, with ** p<0.01, *** p<0.001, ****p<0.0001. FIG. 6D shows plasma insulin levels in 18-month old male CD47^{-/-} and WT C57/BL6 mice after a 6 hour fast (N=6-8 mice/group).

FIG. 7. Shows the consequences of deleting CD47 on the action of injected insulin in WT and CD47^{-/-} mice, and thus compares insulin tolerance between mice groups. FIG. 7A shows how the same volume of insulin is able to function for a significantly longer period in a CD47^{-/-} mice compared to a WT mice. Right panel (FIG. 7B) shows the calculation of the area under curve (AUC) of the glucose measurements. Student's t-test was used to calculate p-value (*p<0.05, **p<0.01).

FIG. 8. Shows how CD47 antagonism improves glycemic control and delays the onset of diabetes in NOD mice and prevents them from getting overt diabetes once they are diabetic. 6-week-old NOD mice (n=10/group) were divided into 3 groups (NOD, IgG, CD47 Ab). NOD group was not treated and acted as a control. The other two groups were injected with a single dose of IgG or MIAP301 (CD47 Ab) 1 h before the onset of the dark phase (1900 hours) and glucose levels were measured at 0900 hours the following day. Right panel shows the corresponding plasma insulin levels, which were measured using ELISA after blood was withdrawn and plasma was isolated from IgG and CD47 ab-treated mice. FIG. 8B shows NOD mice (n=10/group) treated with IgG or MIAP301 once every week and diabetes progression monitored over the course of 21 weeks. When 0.4 μg MIAP301-treated mice eventually crossed diabetic threshold (>10 mmol/1), they were further divided into 3 groups (FIG. 8C): 0.4-treated, 0.8 μg-treated and 1.6 μg-treated and monitored further. FIG. 8D shows the effect of MIAP301 on diabetes in NOD mice. Student's t-test was used to calculate p-value (*p<0.05; **p<0.01).

FIG. 9. Shows the effects of CD47 deletion on insulin secretion in β-cells using transmission electron microscopy (TEM) (FIG. 9A). FIG. 9B compares numbers of docked insulin granules at the β-cell membrane of CD47^{-/-} (CD47-null) mice compared to WT after glucose stimulation. FIG. 9C compares exocytosis of insulin granules from the β-cell membrane of β-cells of CD47^{-/-} mice compared to WT after glucose stimulation. Data are presented as the mean±SEM from the counting of insulin granules in at least two β-cells per mouse, 10 mice per group. Comparisons were made using 2-way ANOVA, followed by Sidak's test for multiple comparisons. A p<0.05 was considered statistically significant (i.e. **p≤0.01; ***p≤0.001).

### DETAILED DESCRIPTION

### General Definitions

The terms “a,” “an,” “the” and similar referents used in the context of describing the invention (especially in the context of the following claims) are to be construed to cover both the singular and the plural, unless otherwise indicated herein or clearly contradicted by context.

All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Except where otherwise indicated, all numbers expressing quantities of ingredients, reaction conditions, and so forth used in the specification and claims are to be understood as being modified in all instances by the term ‘about’. Accordingly, unless indicated to the contrary, the numerical parameters set forth in the following specification and attached claims are approximations that may vary depending upon the desired properties sought to be obtained by the present invention. At the very least, each numerical parameter should be construed in light of the number of significant digits and ordinary rounding conventions. The term “about” may be understood to refer to a range of +/−10%, such as +/−5% or +/−1% or, +/−0.1%.

Recitation of ranges of values herein is merely intended to serve as a shorthand method of referring individually to each separate value falling within the range. Unless otherwise indicated herein, each individual value is incorporated into the specification as if it were individually recited herein. For example, if a range is from about 1 to about 50, it is deemed to include, for example, 1, 7, 34, 46.1, 23.7, or any other value or range within the range.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and preferably together in the same formulation.

As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity.

The terms “therapeutically effective amount” or “therapeutic amount” are intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

The terms “comprise”, “comprises”, “comprised” or “comprising”, “including” or “having” and the like in the present specification and claims are used in an inclusive sense, that is to specify the presence of the stated features but not preclude the presence of additional or further features.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

The term “pharmaceutically acceptable” as used herein refers to substances that do not cause substantial adverse allergic or immunological reactions when administered to a subject. A “pharmaceutically acceptable carrier” includes, but is not limited to, solvents, coatings, dispersion agents, wetting agents, isotonic and absorption delaying agents and disintegrants.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s). The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional. As used herein, “prevent”, “preventing”, “prevention”, or “prophylaxis” of a disease or disorder means preventing that a disorder occurs in subject, and also includes reduction of risk, incidence and/or severity of a condition or disorder.

The phrase “gene silencing” refers to a process by which the expression of a specific gene product is lessened or attenuated. The level of gene silencing can be measured by a variety of means, including, but not limited to, measurement of transcript levels by Northern Blot Analysis, B-DNA techniques, transcription-sensitive reporter constructs, expression profiling (e.g., DNA chips), and related technologies. Alternatively, the level of silencing can be measured by assessing the level of the protein encoded by a specific gene. This can be accomplished by performing a number of studies including Western Analysis, measuring the levels of expression of a reporter protein that has e.g., fluorescent properties (e.g., GFP) or enzymatic activity (e.g., alkaline phosphatases), or several other procedures.

The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region.

A “morpholino oligonucleotide”, also referred to herein as a “morpholino” is an oligonucleotide that comprises an antisense oligonucleotide and a backbone of methylenemorpholine rings linked through phosphorodiamidate groups. Antisense morpholinos, typically 18-25 nucleotides in length, can be designed to bind to a complementary sequence in a selected mRNA to modulate gene expression.

As used herein, the term “isolated” when used in connection with cells or nucleic acid molecules means isolation from the environment in which the cell/molecule normally exists in nature. Isolation may involve separation of the cell/molecule from other cellular/molecular material, or medium when produced by recombinant techniques.

As used herein, “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogues, or polynucleotide analogues that are biologically synthesised by heterologous systems, as well as proteins and/or mRNAs encoded by such nucleic acids. Thus, for example, a protein synthesised by a microorganism is recombinant, for example, if it is synthesised from an mRNA synthesised from a recombinant gene present in the cell.

### Methods of Preparing Transplantable Pancreatic Islet Beta Cells

CD47 (Cluster of Differentiation 47, also referred to as integrin associated protein or ‘IAP’) is a transmembrane protein that, in humans, is encoded by the CD47 gene, and which binds the ligands thrombospondin-1 (TSP-1) and signal-regulatory protein alpha (SIRPα) (ISRN Hematol. 2013: 614619).

CD47 expression can be seen in various tissues and cell types throughout the body, ranging from microglia to red blood cells. Wide expression suggests that CD47 is involved in a range of cellular processes, including apoptosis, proliferation, adhesion, and migration. Furthermore, it plays a key role in immune and angiogenic responses. CD47 is ubiquitously expressed in human cells, and under normal circumstances, CD47 acts as a signal that prevents white blood cells (ie macrophages) attacking the body's own cells and tissues (Science. 2000 Jun. 16; 288(5473):2051-4). The fact that CD47 has been found to be overexpressed in many different tumour cells has made it a promising target in anti-cancer therapy (Curr Opin Immunol. 2012 April; 24(2): 225-232).

CD47 and TSP-1 have also been shown to have relevance in obesity. In one study, investigators examined the effect of TSP-1 deficiency on the development of obesity in a high fat diet induced obese mouse model. While TSP-1 deficient mice were not protected against obesity, it was found that a TSP1 deletion reduces inflammation and improves whole body insulin sensitivity in the obese state (PLoS One; Vol. 6, Iss. 10: e26656). A further study by these investigators ascertained whether CD47 plays a role in the development of obesity and metabolic complications. In contrast to the TSP-1 deletion study, it was found that CD47 deficient mice were protected from high fat diet-induced obesity, displaying decreased weight gain and reduced adiposity. Similarly, to the TSP-1 deletion study, CD47 deficient mice showed reduced inflammation which correlated with improved glucose tolerance and insulin sensitivity in connection with reduced obesity.

The present invention is predicated on the surprising and unexpected finding that CD47 influences insulin production in pancreatic islet beta cells. That is, it has been found that a reduction of CD47 in non-diabetic pancreatic beta islet cells increases insulin expression in said cells.

According to one embodiment, the mRNA coding for CD47 is mammalian, preferably human. In another embodiment, siRNA and morpholinos are used to specifically target and reduce the expression of CD47.

In exemplary embodiments, mRNA sequences coding for human CD47 useful as a target for siRNA-mediate silencing may be selected from:

| Name | NCBI Reference | SEQ ID NO. |
|---|---|---|
| Homo sapiens CD47 Molecule, | NM_001777.4 | 1 |
| transcript variant 1 | | |
| Homo sapiens CD47 Molecule, | NM_198793.3 | 2 |
| transcript variant 2 | | |

In exemplary embodiments, mRNA sequences coding for human CD47 useful as a target for siRNA-mediate silencing may be selected from:

| Name | NCBI Reference | SEQ ID NO. |
|---|---|---|
| Mus musculus CD47 Molecule, | NM_001368415.1 | 3 |
| transcript variant 1 | | |
| Mus musculus CD47 Molecule, | NM_001368416.1 | 4 |
| transcript variant 2 | | |

In exemplary embodiments, siRNA sequences useful in silencing human CD47 expression may be selected from:

| | | SEQ |
|---|---|---|
| Name | SEQ | ID NO. |
| Human CD47 siRNA-1 | 5′-CCU CUU UGA AGA UGG | 5 |
| | AUA ATT-3′ | |
| Human CD47 siRNA-2 | 5′-AAG ATG GAT AAG AGT | 6 |
| | GAT GCT CCT GTC TC-3′ | |

In exemplary embodiments, siRNA sequences useful in silencing murine CD47 expression may be selected from:

| | | SEQ |
|---|---|---|
| Name | SEQ | ID NO. |
| Murine CD47 siRNA-1 | 5′-GAA UGA CCU CUU UCA | 7 |
| | CCA-3′ | |
| Murine CD47 siRNA-2 | 5′-UUA UCC AUC UUC AAA | 8 |
| | GAG GTT-3′ | |

In further exemplary embodiments, morpholino sequences useful in silencing human and murine CD47 expression may include:

| | | SEQ |
|---|---|---|
| Name | SEQ | ID NO. |
| Antisense oligonucleotide | CGTCACAGGCAGGA | 9 |
| to human CD47 | CCCACTGCCCA | |
| Antisense oligonucleotide | AACAGGCAAACTGT | 10 |
| to mouse CD47 | GTCACTTACCC | |

### Gene Silencing Through RNA Interference

RNA has been used for several years to reduce or interfere with expression of targeted genes in a variety of systems. Although originally thought to require use of long double-stranded RNA (dsRNA) molecules, the active mediators of RNA interference (RNAi) are now known to be short dsRNAs. Short single-stranded antisense RNA molecules were demonstrated to be effective inhibitors of gene expression more than a decade ago but are susceptible to degradation by a variety of nucleases and are therefore of limited utility without chemical modification. Double-stranded RNAs are surprisingly stable and, unlike single-stranded DNA or antisense RNA oligonucleotides, do not need extensive modification to survive in tissue culture media or living cells.

Short interfering RNAs are naturally produced by degradation of long dsRNAs by Dicer, an RNase Ill class enzyme. While these fragments are usually about 21 bases long, synthetic dsRNAs of a variety of lengths, ranging from 18 bases to 30 bases (Nature Biotechnology. 23: 222-226), can be used to suppress gene expression. These short dsRNAs are bound by the RNA Induced Silencing Complex (RISC), which contains several protein components including a ribonuclease that degrades the targeted mRNA. The antisense strand of the dsRNA directs target specificity of the RISC RNase activity, while the sense strand of an RNAi duplex appears to function mainly to stabilize the RNA prior to entry into RISC and is degraded or discarded after entering RISC.

Short (18-30 bp) RNA duplexes, when introduced into mammalian cells, can produce sequence-specific inhibition of target mRNA can be realized without inducing an interferon response. Certain of these short dsRNAs, referred to as small inhibitory RNAs (“siRNAs”), can act catalytically at sub-molar concentrations to cleave greater than 95% of the target mRNA in the cell (EMBO J. 21(21): 5864-5874).

From a mechanistic perspective, introduction of long double stranded RNA into plants and invertebrate cells is broken down into siRNA by a Type Ill endonuclease known as Dicer (Genes Dev. 15:485). Dicer, a ribonuclease-Ill-like enzyme, processes the dsRNA into 19-23 base pair short interfering RNAs with characteristic two base 3′ overhangs. The siRNAs are then incorporated into a RISC where one or more helicases unwind the siRNA duplex, enabling the complementary antisense strand to guide target recognition (Cell 107:309). Upon binding to the appropriate target mRNA, one or more endonucleases within the RISC cleaves the target to induce silencing (Genes Dev. 15:188).

The interference effect can be long lasting and may be detectable after many cell divisions. Moreover, RNAi exhibits sequence specificity. (J. Biochem. 363:1-5). Thus, the RNAi machinery can specifically knock down one type of transcript, while not affecting closely related mRNA. These properties make siRNA a potentially valuable tool for inhibiting gene expression and studying gene function and drug target validation. Moreover, siRNAs are potentially useful as therapeutic agents against: (1) diseases that are caused by over-expression or misexpression of genes; and (2) diseases brought about by expression of genes that contain mutations.

An siRNA useful in the present invention is considered to completely inhibit CD47 expression or activity when the level of CD47 expression or activity in the presence of CD47-specific siRNA is decreased by at least 95%, preferably by 96%, 97%, 98%, 99% or 100% as compared to the level of CD47 expression or activity in the absence of specific inhibition. An siRNA useful in the present invention is considered to significantly inhibit CD47 expression or activity when the level of CD47 expression or activity in the presence of CD47-specific siRNA is decreased by at least 50%, preferably, 55%, 60%, 75%, 80%, 85% or 90% as compared to the level of CD47 expression or activity in the absence of binding with a CD47 antibody described herein. In one embodiment, the effective inhibition of CD47 expression or activity necessary to observe effects on insulin expression or secretion is a minimum of a 50% reduction to a maximum of a 100% reduction in CD47 expression levels by using CD47-targetting siRNA. Preferably, the percentage reduction is determined by measuring relative density of SDS-PAGE gel bands (J Immunol Methods. 2018 June; 457:1-5).

### CD47 Blocking Antibody

A full-length antibody as it exists naturally is an immunoglobulin molecule comprising two heavy (H) chains and two light (L) chains interconnected by disulfide bonds. The amino terminal portion of each chain includes a variable region of about 100-110 or more amino acids primarily responsible for antigen recognition via the complementarity determining regions (CDRs) contained therein. The carboxy-terminal portion of each chain defines a constant region primarily responsible for effector function.

The CDRs are interspersed with regions that are more conserved, termed framework regions (“FR”). Each light chain variable region (LCVR) and heavy chain variable region (HCVR) is composed of 3 CDRs and 4 FRs, arranged from amino-terminus to carboxy-terminus in the following order: FR1, CDR1, FR2, CDR2, FR3, CDR3, FR4. The 3 CDRs of the light chain are referred to as “LCDR1, LCDR2, and LCDR3” and the 3 CDRs of the heavy chain are referred to as “HCDR1, HCDR2, and HCDR3.” The CDRs contain most of the residues which form specific interactions with the antigen. The numbering and positioning of CDR amino acid residues within the LCVR and HCVR regions are in accordance with the well-known Kabat numbering convention. While the light chain CDRs and heavy chain CDRs disclosed herein are numbered 1, 2, and 3, respectively, it is not necessary that they be employed in the corresponding antibody compound light and heavy chain variable regions in that numerical order, i.e., they can be present in any numerical order in a light or heavy chain variable region, respectively.

Light chains are classified as kappa or lambda, and are characterised by a particular constant region as known in the art. Heavy chains are classified as gamma, mu, alpha, delta, or epsilon, and define the isotype of an antibody as IgG, IgM, IgA, IgD, or IgE, respectively. IgG antibodies can be further divided into subclasses, e.g., IgG1, IgG2, IgG3, IgG4. Each heavy chain type is characterized by a particular constant region with a sequence well known in the art.

The monoclonal antibodies and other antibody compounds useful in the methods and compositions described herein can be any of these isotypes. Further, such antibodies and other antibody compounds include fully human monoclonal antibodies, as well as humanised monoclonal antibodies and chimeric antibodies, in addition to functional (e.g. binding) fragments of said antibodies.

According to embodiments, CD47 antibodies useful for performing the present invention exhibit inhibitory activity, for example by inhibiting CD47 expression (e.g. inhibiting cell surface expression of CD47), or function, or by interfering with the interaction between CD47 and SIRPα. The antibodies provided herein completely or partially reduce or otherwise modulate CD47 expression or function upon binding to, or otherwise interacting with, CD47, preferably a human CD47. The reduction or modulation of a biological function of CD47 is complete, significant, or partial upon interaction between the antibodies and the CD47 polypeptide.

In embodiment, an antibody useful in the present invention is considered to completely inhibit CD47 function (e.g. by blocking ligands binding) when the level of CD47 function in the presence of the antibody is decreased by at least 95%, preferably by 96%, 97%, 98%, 99% or 100% as compared to the level of CD47 function in the absence of interaction (e.g. binding) with an antibody described herein. In other embodiments, a CD47 antibody is considered to significantly inhibit CD47 function when the level of CD47 function in the presence of the CD47 antibody is decreased by at least 50%, preferably, 55%, 60%, 75%, 80%, 85% or 90% as compared to the level of CD47 function in the absence of binding with a CD47 antibody described herein. In other embodiments, an antibody is considered to partially inhibit CD47 function when the level of CD47 function in the presence of the antibody is decreased by less than 50%, preferably, 10%, 20%, 25%, 30%, or 40% as compared to the level of CD47 function in the absence of binding, with an antibody described herein. In one embodiment, the effective minimum concentration of CD47-blocking antibody required to achieve observed effects on insulin expression or secretion is 10 ug/ml (Nature volume 536, pages 86-90 (2016)).

In a preferred embodiment, a CD47 antibody useful in performing the present invention may be selected from the group consisting of anti-CD47 monoclonal antibodies (clone B6H12, sc-12730, Santa Cruz Inc. CA, USA and/or clone D307P, #63000, Cell Signalling Inc. MA, USA).

### Gene Silencing Mediated by Morpholino Oligonucleotides

Morpholinos function by an RNase H-independent mechanism (i.e., a steric block mechanism as opposed to an RNase H-cleavage mechanism) and are soluble in aqueous solutions (Nat. Genet 26:216-220; Annu. Rev. Pharmacol. Toxicol. 4:403-19). Further, Morpholinos are generally are stable in cells because their morpholine backbone is not recognized by nucleases, and they can be highly effective with predictable targeting.

In embodiments, a morpholino suitable for the present invention can be between about 7 and 100 nucleotides long, between 10 and 50, between 20 and 35, and between 15 and 30 nucleotides long. In a preferred embodiment, the morpholino oligonucleotide is between about 18 and about 25 nucleotides long. The oligonucleotides can be 17, 18, 19, 20, 21, 22, 23, 24 or 25 nucleotides long.

The morpholino molecule can be designed such that every residue is complementary to a residue in the target molecule. Alternatively, one or more substitutions can be made within the molecule to increase stability and/or enhance processing activity of said molecule. Substitutions can be made within the strand or can be made to residues at the ends of the strand.

In a preferred embodiment, a translation-blocking antisense morpholino oligonucleotide complementary to human and murine CD47 consists of the sequence: CGTCACAGGCAGGACCCACTGCCCA (SEQ ID NO: 7).

In embodiments, a morpholino considered useful in the present invention may completely inhibit CD47 expression or activity when the level of CD47 expression or activity in the presence of the CD47-specific morpholino is decreased by at least 95%, preferably by 96%, 97%, 98%, 99% or 100% as compared to the level of CD47 expression or activity in the absence specific inhibition. In other embodiments, a morpholino considered useful in the present invention may significantly inhibit CD47 expression or activity when the level of CD47 expression or activity in the presence of the CD47-specific morpholino is decreased by at least 50%, preferably, 55%, 60%, 75%, 80%, 85% or 90% as compared to the level of CD47 expression or activity in the absence of specific inhibition. In other embodiments, a morpholino considered useful in the present invention may partially inhibit CD47 expression or activity when the level of CD47 expression or activity in the presence of the CD47-specific morpholino is decreased by less than 50%, preferably, 10%, 20%, 25%, 30%, or 40% as compared to the level of CD47 expression or activity in the absence of specific inhibition. In one embodiment, the effective inhibition of CD47 expression or activity required to observe effects on insulin expression or secretion is a minimum of a 50% reduction to a maximum of a 100% reduction in CD47 expression levels by using CD47-targetting morpholino. The percentage reduction is determined by measuring relative density of SDS-PAGE gel bands (J Immunol Methods. 2018 June; 457:1-5).

Methods of Use and/or Therapy

In another aspect, the invention provides methods of administering CD47-specific silencing molecule to a subject (e.g. a cell, tissue or organism). Further, administration may be in vitro, in vivo or ex vivo. The silencing molecule is preferably the CD47-specific siRNA, blocking antibody or morpholino, or combination thereof, as described above.

In embodiments, the method of administration generally includes administering a biologically effective amount of a composition comprising CD47-specific silencing molecule composition to a subject. The language “biologically effective amount” is an amount necessary or sufficient to produce a desired physiologic response. The effective amount may vary depending on such factors as the size and weight of the subject, or the particular compound/molecule being administered. Administration may be by any route, for example, injection intravenously, intradermally, subcutaneously, or by suitable delivery vector (nucleic acid vaccine, virus or bacteria).

The administration target may be a cell, a cell culture, an organ, a tissue etc. For example, in one embodiment, the CD47-specific silencing molecule to a cell isolated from the host. Preferably, the cells is a pancreatic islet beta cell.

Pharmaceutical compositions useful for delivering a CD47-specific silencing molecule according to the invention may include said molecule and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. These pharmaceutical compositions can be included in kits, such as, for example, diagnostic kits.

Gene therapy presents another therapeutic alternative for achieving CD47-specific silencing in a host. The introduction and expression of transgenes in pancreatic islets prevent immune rejection and improve proliferation and survival of islet grafts has been the focus of much research (e.g. Diabetes Metab Res Rev. 22(3):241-52; Endocr Dev. 12:24-32; Diabetes. 54 Suppl 2:S87-96). Transgene delivery via ex-vivo transduction of human islets has also been investigated (Journal of Biol Chem. 278:343-351; Transplantation Proceedings, 39:3436-3437). Both viral and non-viral vectors may be used as carriers for effective and safe delivery of transgenes (Int. J. Mol. Sci. 2019, 20(21), 5491) as well as inhibitory RNA (Methods Mol Biol. 1950:3-18). Such methods may be adapted to achieve a reduction CD47 expression in host that is the recipient of a pancreatic islet beta cell transplant, thereby increasing insulin expression in said transplanted cells.

In embodiments, a modified pancreatic islet beta cell in which CD47 expression has been reduced may have comparable or negligibly higher, or at most 2 or 3 fold higher insulin expression and/or secretion, when compared to an unmodified pancreatic islet beta cell in the absence of glucose stimulation. In embodiments, in the presence of glucose stimulation, a modified pancreatic islet beta cell in which CD47 expression has been reduced may have increased insulin expression and/or secretion at least 3 fold, preferably at least 5 fold, more preferably at least 10 fold, when compared to an unmodified pancreatic islet beta cell stimulated with glucose.

The transplantable beta islet cells prepared according to the present invention may be used to treat any disease or disorder caused or associated with defective insulin production, a defective ability to utilise insulin or defective insulin signalling. In embodiments, the disease or disorder may be selected from type 1 diabetes, type 2 diabetes, gestational diabetes, congenital diabetes due to genetic defects of insulin secretion, cystic fibrosis-related diabetes, steroid diabetes induced by high doses of glucocorticoids, monogenic diabetes, impaired glucose tolerance, hyperglycaemia or metabolic syndrome.

The modified pancreatic islet cells of the invention may be transplanted into a subject in need using any means known in the art, including, but not limited to, introduction via the recipient's portal vein, under the renal capsule, into the sternomastoid muscle, intraperitoneally, in the gastric submucosa, in the testes, or in the spleen (Cell Transplantation, 15:89-104, Transplantation, 86:753-760, Cell Transplant. 17(9):1005-14). Transplantation also includes xenotransplantation, in which pancreatic islet cells are derived from a non-human source, preferably a porcine animal.

The invention also provides for the treatment of diabetes, generally, in a subject in need. In embodiments, a method for treating diabetes comprises modulating CD47 expression or function in a subject (including cells derived therefrom). Preferably, treatment comprises administering to said subject an effective amount of a CD47-specific silencing molecule, further preferably wherein said CD47-specific silencing molecule is a CD47-specific siRNA, blocking antibody or morpholino.

Further examples of the invention are described below. However, it should be noted that the invention should not be limited to these examples, and that the invention is susceptible to variations, modifications and/or additions other than those specifically described, and it is to be understood that the invention includes all such variations, modifications and/or additions which fall within the scope of the claims.

### EXAMPLES

### Example 1—Reducing CD47 Expression Improves Glucose Homeostasis and Glucose-Stimulated Insulin Secretion

An experiment was performed to determine the effect of CD47 blockade on glucose homeostasis in mice. Three-month-old wildtype (WT) C57/BL6 mice (Australian Bioresources, Moss Vale, NSW, Australia) were fasted for 4 hours, then injected intraperitoneally with either IgG (sc-3883, Santa Cruz Inc.; control) or anti-CD47 antibody (clone MIAP301, SC-12731, Santa Cruz Inc.) at a concentration of 0.4 μg/g body weight of mice. Two hours after the injection of antibody, an intraperitoneal glucose tolerance test (IPGTT) was conducted. Briefly, a tail cut was made, a blood sample taken and fasting blood glucose level (BGL) measured using a Stat Strip Glucometer (NovaBiomedical, UK). A time zero sample was taken, followed by the administration of a glucose bolus (2 g/kg body weight) via intraperitoneal injection, and BGL measured at 5, 10, 15, 20, 30, 45, 60, and 120 minutes post-injection. In parallel, blood was collected at these different time-points and plasma was collected by centrifugation, and insulin levels measured using ELISA.

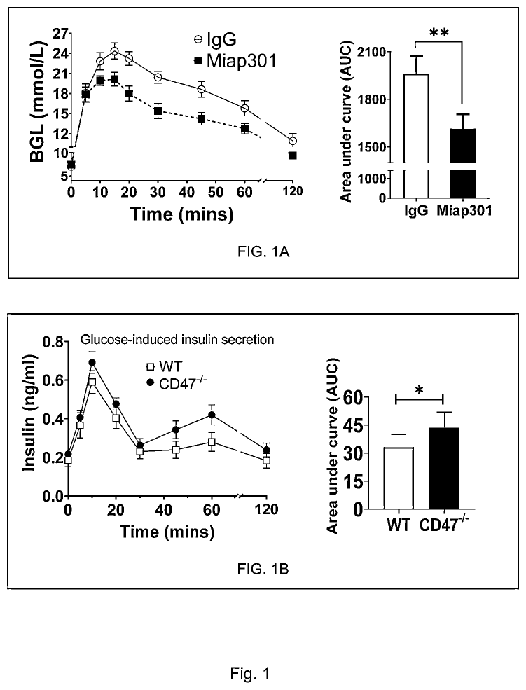

FIG. 1 shows the results of this experiment. The left panel in FIG. 1A shows that a 2-hour treatment of mice with MIAP301 prevented the rise in blood glucose level compared to the control (N=10 mice/group). The right panel shows the calculation of the area under curve (AUC) of the glucose measurements, and a Student's t-test was used to calculate p-value. As can be seen, mice treated with MIAP301 had significantly increased glucose tolerance (p<0.001) compared to the corresponding control group.

### Example 2—Reducing CD47 Expression or Activity in Pancreatic Beta Islet Cells Increases Insulin Protein Content

Experiments were performed to determine the effect of reducing CD47 gene expression on insulin gene expression in murine and human-derived pancreatic beta islet cells.

Murine pancreatic beta islet cells were (AddexBio Technologies Inc, MIN6), and human pancreatic beta islet cells were isolated from cadaveric donors at Westmead hospital in Sydney, Australia. Cells were grown in cell culture media consisting of Dulbecco's modified eagle media (DMEM), complimented with 15% FBS, 1% P/S, 1% Glutamine (2 mM), 20 mM HEPES and 50-55 μM beta-mercaptoethanol). Cells were passaged every 3-4 days and media was changed every 2 days.

For siRNA knockdown of CD47 expression, the Silencer™ siRNA Construction kit (and components thereof) were sourced from Ambion (Catalog #1620; Austin, TX), which employs a T7 promoter to produce dsRNAs was used. For human CD47, sequences SEQ ID NO. 5 and SEQ ID NO. 6 were produced. For murine CD47, sequences SEQ ID NO. 7 and SEQ ID NO. 8 were produced. Murine and human cultured islet cells were starved for one hour in media (DMEM, 2% FBS) before incubating for 48 hours at a concentration of 10 nM CD47 siRNA molecules. After 48 hours, cells were lysed with lysis buffer (RIPA, Cell Signalling Tech Inc, MA, USA) and subjected to SDS-PAGE.

A translation-blocking antisense morpholino oligonucleotide complementary to human and murine CD47 (CGTCACAGGCAGGACCCACTGCCCA) (SEQ ID NO: 9) and a 5-base mismatch control (CGTGACAGCCACGACCGACTGCGCA) (SEQ ID NO: 11) were obtained from GeneTools (Philomath, Oregon). Human cultured islet cells were treated with the morpholinos (10 μmol/L) for 48 hours. After 48 hours, cells were lysed with lysis buffer (RIPA, Cell Signalling Tech Inc, MA, USA) and subjected to SDS-PAGE.

An anti-CD47 blocking antibody (clone MIAP301, SC-12731, Santa Cruz Inc.) was also used to block CD47 function.

Pancreatic beta islet cells (MIN6) with indicated gene knockdown were seeded at 2×10⁵ cells/well in 24-well plates in DMEM cell media, complimented with 15% FBS, 1% P/S, 1% Glutamine (2 mM), 20 mM HEPES and 50-55 μM beta-mercaptoethanol). Next morning, the media was removed, and the cells were washed with PBS twice. Prior to the insulin secretion assay, the cells were starved for 30 min in Henseleit-Krebs-Ringer buffer (HKRB, 119 mM NaCl, 4.74 mM KCl, 2.54 mM CaCl₂, 1.19 mM MgCl₂, 1.19 mM KH2PO4, 25 mM NaHCO₃, 10 mM HEPES, pH 7.4) supplemented with 0.2% bovine serum albumin (BSA) and 2 mM glucose. The cells were then incubated for 60 min in HKRB containing 2 mM or 20 mM glucose to measure glucose-stimulated insulin secretion.

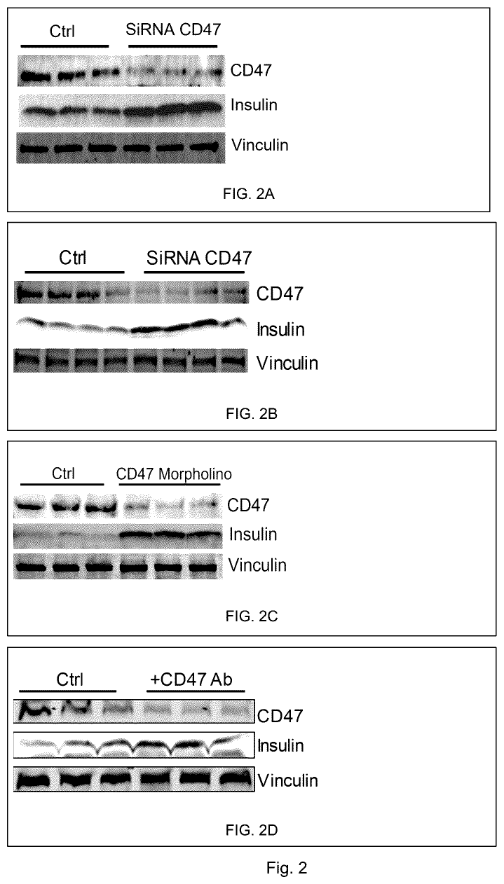

FIG. 2A and FIG. 2B show that reducing CD47 gene expression by siRNA knockdown has the effect of increasing insulin protein content in glucose stimulated human and murine pancreatic beta islet cells, respectively, relative to housekeeping gene vinculin. The right panels of FIGS. 2A and 2B shows a relative density comparison—the panels respectively show that human and murine beta islet cells treated with siRNA contained approximately 4-fold more insulin in comparison to the control.

FIG. 2C shows that reducing CD47 gene expression using an anti-CD47 morpholino knockdown had the effect of increasing insulin protein content in glucose stimulated human pancreatic beta islet cells relative to vinculin expression. The right panel of FIG. 2C shows approximately a 4 fold relative increase in protein content in morpholino treated cells in comparison to the control.

FIG. 2D shows that reducing CD47 expression by way of an anti-CD47 blocking antibody also had the effect of increasing insulin protein content in glucose stimulated mouse pancreatic beta islet cells relative to vinculin expression. The right panel of FIG. 2D shows that morpholino treated cells had at least a 2 fold relative increase in insulin protein content compared to the control.

### Example 3—Reducing CD47 Expression in Pancreatic Beta Islet Cells Increases Insulin Protein Production and Secretion

An experiment was performed to determine the effect of reducing CD47 on insulin production and secretion in vitro.

Murine (MIN6, AddexBio Technologies Inc, San Diego, CA, USA) and human pancreatic beta islet cells (isolated from cadaveric donors at Westmead hospital in Sydney, Australia) were seeded at 2×10⁵ cells/well in 24-well plates in DMEM cell media, complimented with 15% FBS, 1% P/S, 1% Glutamine (2 mM), 20 mM HEPES and 50-55 μM beta-mercaptoethanol). RNA interference was performed for 48 hrs according to sequences and procedure of Example 2. Specifically, the next day the media was removed and cells were washed 2× with PBS. Cells were starved for an hour in starvation media (DMEM+2% FBS). Murine cells were incubated with siRNA targeting CD47 while human islets were incubated with CD47-targetting Morpholino for 48 hours. After incubation, prior to the insulin secretion assay, the cells were starved for 30 min in Henseleit-Krebs-Ringer buffer (HKRB, 119 mM NaCl, 4.74 mM KCl, 2.54 mM CaCl₂, 1.19 mM MgCl₂, 1.19 mM KH₂PO4, 25 mM NaHCO₃, 10 mM HEPES, pH 7.4) supplemented with 0.2% bovine serum albumin (BSA) and 2 mM glucose. The cells were then incubated for 60 min in HKRB containing 2 mM or 20 mM glucose to measure glucose-stimulated insulin secretion. The supernatant medium was collected, and the secreted insulin was measured using an ELISA kit (Mercodia, Uppsala, Sweden). Insulin concentration of the supernatant was normalized to the total protein of the cells. The insulin secretion rate was expressed as the amount of secreted insulin (ng/μg protein)/h (Diabetologia. 1993; 36(11):1139-1145; PLoS One. 2016; 11(3):e0151927; Cell. 2007; 129(2):359-370).

For cellular insulin protein content, the islet cells in each well were washed twice with PBS, trypsinized and centrifuged. Trypsin was completely removed, and 0.1 ml water was added to the collected cells (approx. 40.000 cells). Cells were sonicated for 10 seconds (30% amplitude) and mixed with equal amount of acid-ethanol (0.18 M HCl in 75% ethanol) and incubated overnight at −20° C. Acid-Ethanol extract was neutralized with 100 μl 1 M Tris pH 7.5; followed by centrifugation (1×10⁴ g, 10 min) and supernatant was collected and ELISA was used to measure the insulin concentration in the islets. To the other aliquots of cells, RIPA buffer (Cell Signalling Technology) was added and total protein concentration was determined using BCA assay. The insulin content was expressed as the amount of insulin (ng) per μg protein. The insulin protein expression was examined using SDS-PAGE and normalized to a housekeeping gene vinculin.

In terms of statistical analysis, an Anova was performed followed by Tukey's multiple comparison test was used to calculate p-value, with *p<0.05, ** p<0.01, *** p<0.001.

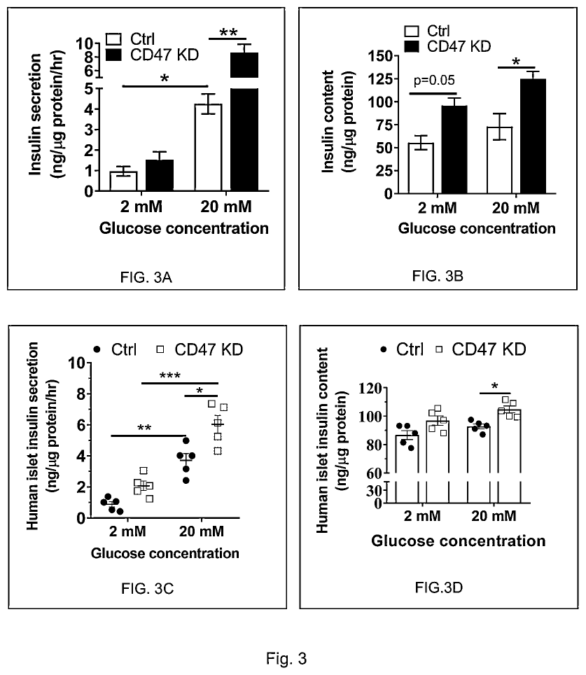

FIG. 3A shows changes in glucose-stimulated insulin secretion (GSIS) from mouse pancreatic islet cells, achieved by reducing CD47 levels by siRNA treatment. Insulin secretion was markedly improved after glucose stimulation in CD47-depleted cells relative to a control (scrambled siRNA). FIG. 3B shows the difference in insulin protein content in mouse pancreatic islet cells, achieved by manipulating CD47 levels using siRNA. Surprisingly, CD47-deficient cells were found to have higher insulin content with or without glucose stimulation relative to a control.

FIG. 3C shows changes in GSIS from human pancreatic islets, achieved by reducing CD47 levels using a CD47 morpholino. GSIS is higher in human islets where CD47 expression has been decreased. FIG. 3D shows the difference in insulin protein content in human pancreatic islets, achieved by manipulating CD47 levels using a CD47 morpholino. Similarly to the observations with murine cells, human pancreatic beta islet cell insulin protein content increased where CD47 levels were reduced with CD47 morpholino treatment.

### Example 4—Reducing CD47 Expression in Pancreatic Beta Islet Cells Improves Transplantation Outcomes in Diabetic Mice

Experiments were performed to determine the effect of CD47 depletion on glycemic control in diabetic mice transplanted with pancreatic beta islet cells. In a first experiment, streptozotocin-induced diabetic C57/BL6 mice (3 months old; Australian Bioresources, Moss Vale, NSW, Australia) were transplanted with equal number of islets isolated from wildtype (WT) mice or an engineered equivalent lacking CD47 (CD47-null mice; Australian Bioresources, Moss Vale, NSW, Australia).

In a second experiment, streptozotocin-induced diabetic C57/BL6 mice (3 months old; Australian Bioresources, Moss Vale, NSW, Australia) were transplanted with equal number of islets isolated from wildtype (WT) mice with CD47 blocked using an antibody (MIAP301 as described above) compared to isotype-matched control IgG (sc-3883, Santa Cruz Inc.). To achieve blockade, isolated islets from WT mice were incubated for 30-45 minutes in an incubator (37 degree, 5% carbon dioxide) with MIAP301 or isotype-matched control IgG at 10 ug/ml before transplantation.

In both experiments, blood glucose levels (BGL) were then monitored for the next 20 days.

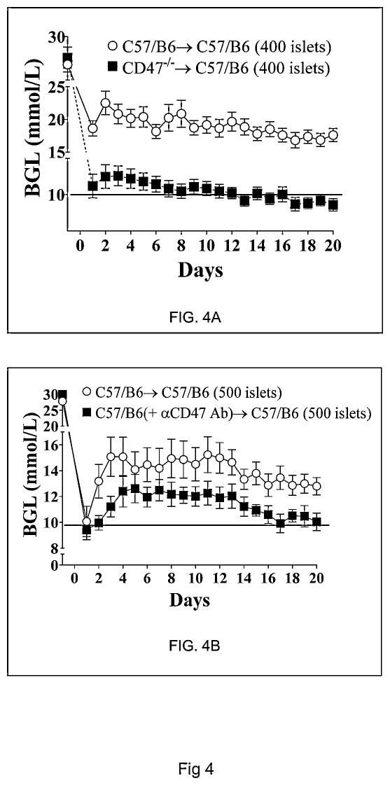

FIG. 4A shows the difference in islet transplantation efficiency and glycemic control achieved in diabetic mice transplanted with equal number (400) non-diabetic WT donor islets or islets from CD47^{-/-} mice (3 months old; Australian Bioresources, Moss Vale, NSW, Australia). A horizontal line represents the average normal blood glucose level (BGL) of these mice when non-diabetic. While transplantation of 400 WT islets were insufficient to reduce BGL to a normal, non-diabetic level in diabetic mice, it was surprisingly found that transplanting CD47^{-/-} islets reduced BGL in diabetic mice to a normal level.

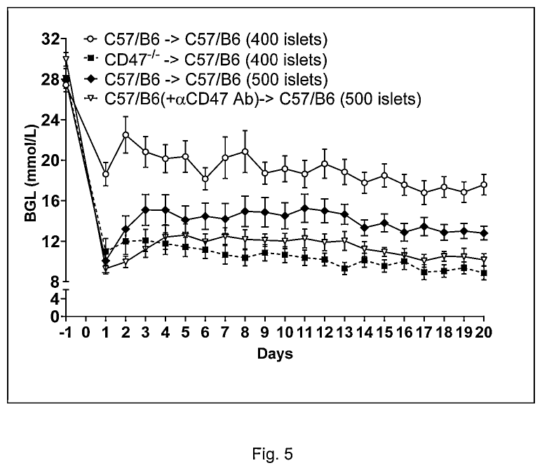

FIG. 4B shows improvement in islet transplantation efficiency and glycemic control achieved by manipulating CD47 levels therapeutically by pre-treating donor islets with MIAP301 versus a control IgG. Surprisingly, diabetic mice transplanted with islets pre-treated with MIAP301 exhibited a reduced BGL compared to diabetic mice transplanted with islets pre-treated with non-specific IgG. FIG. 5 represents the side-by-side comparison of overall islet transplant outcomes—that is, the figure combines the data shown in FIG. 4A and FIG. 4B. Overall, it was found that mice transplanted with islets from CD47^{-/-} mice or islets pre-treated with MIAP301 had better glycemic control than diabetic mice transplanted with WT islets.

### Example 5—Impact of CD47 Deletion in Aged Mice Fed a High Fat Diet

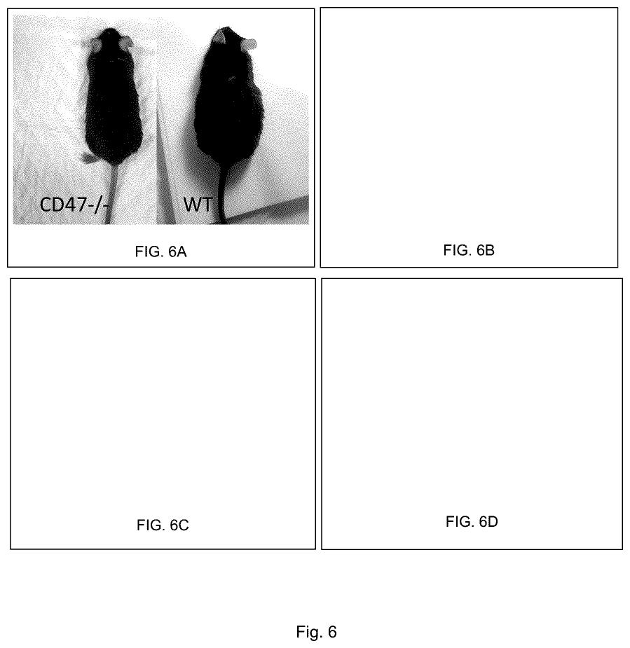

Experiments were performed to determine the impact of CD47 deletion in aged mice fed a high fat diet. CD47^{-/-} and WT C57/BL6 mice (N=10 mice/group) were fed with a standard diet (SD; containing 8% calories from fat; (Gordon's Specialty Stockfeeds, Yanderra, Australia) until 14 months of age, followed feeding each group with a high fat diet (HFD; containing lard/sucrose (45% calories from fat, based on rodent diet D12451; (Research Diets, New Brunswick, NJ, USA) and monitoring weight until 18 months of age. FIG. 6A is a photograph of 18-month old CD47^{-/-} and WT C57/BL6 mice, fed under this protocol. As can be seen, WT mice were larger and showing characteristics of obesity, whereas CD47^{-/-} had a more leaner appearance. FIG. 6B shows prevention of HFD-induced weight gain with age by deleting CD47 in mice and the average body weight differences between the two groups of mice at different ages. Ordinary one-way Anova with multiple comparisons was used to calculate p-value, with *p<0.05.

FIG. 6C shows the blood glucose level (BGL) in 18-month old male CD47^{-/-} and WT C57/BL6 mice after a 6 hour fast (N=10-12 mice/group). FIG. 6D shows plasma insulin levels in 18-month old male CD47^{-/-} and WT C57/BL6 mice after a 6 hour fast (N=6-8 mice/group). Two-way Anova with multiple comparisons was used to calculate p-value, with ** p<0.01, *** p<0.001, ****p<0.0001.

As can be seen in FIG. 6C, BGLs in CD47^{-/-} aged mice do not rise to the same extent as seen in WT when both groups of mice were placed on HFD. This shows that mice lacking CD47 tolerate the stress of HFD better and achieve better glycemic control. Lower plasma insulin levels were noted in aged CD47^{-/-} mice under HFD. While this may appear counterintuitive, the reason for this is likely because of improved glucose homeostasis and glycemic control; since glucose levels do not rise compared to WT mice, there is no need for the beta islet cells to carry higher than necessary insulin constantly.

### Example 6—Impact of CD47 Deletion in Aged Mice Injected with Insulin

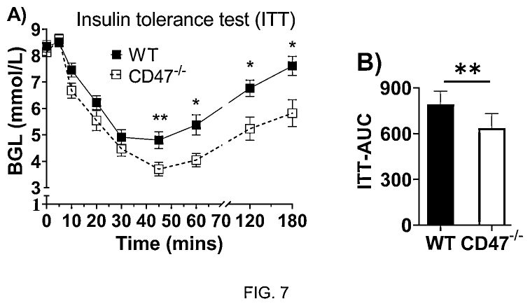

Experiments were performed to determine the impact of CD47 deletion on insulin tolerance in 12-month old (aged) WT (C57/BL6) and CD47^{-/-} mice (N=N=10 mice/group), with the results shown in FIG. 7. Mice were fasted for 6 h and fasting BGL measured, followed by the administration of an insulin bolus (0.75-1 U/kg body weight) by injection intraperitoneally and BGL measurements at 5, 10, 20, 30, 45, 60, 120 and 180 min post-injection. The right panel of FIG. 7 provides the calculation of the area under curve (AUC) of glucose measurements, where a Student's t-test was used to calculate p-value (**p<0.01).

In WT mice, the effect of insulin starts diminishing at 45^{th} minute post-injection and BGL reverts back close to normal level (approx. 8 mmol/L) at around 180 minutes. But the same quantity of insulin works for longer period in CD47^{-/-} mice as BGL is still lower than 7 mmol/L at 180 minutes. CD47 deletion sensitizes the mice to insulin. This shows that CD47 blocking can have similar effects to sulphonylureas class of medication, currently available in market to treat type II diabetes. Sulphonylureas are a class of oral (tablet) medications that control blood sugar levels in patients with type 2 diabetes by stimulating the production of insulin in the pancreas and increasing the effectiveness of insulin in the body.

### Example 7—CD47 Antagonism in NOD Mice

Due to the success of MIAP301 in controlling blood glucose in mice transplanted with a minimal number of mouse islets, it was determined whether this would also be effective in other settings with reduced insulin secretory capacity. Specifically, it was determined whether CD47 receptor blockade could extend the period of normoglycemia in autoimmune prone non-obese diabetic (NOD) mice that develop diabetes spontaneously without any intervention. Abnormalities of β-cell function can be detected in NOD mice before they develop diabetes and this is also true in humans who have dysglycemia before they develop type 1 diabetes (T1 D). Similarly, following diagnosis of T1 D, there is a period with reduced β-cell function that sometimes is still sufficiently effective for insulin treatment to be ceased for a period of time, which is called the ‘honeymoon period’. It was determined whether CD47 blocking would improve glycemic control in NOD mice prior to diabetes with potential application to reducing dysglycemia before and after diagnosis of diabetes and possibly prolonging the time to diagnosis of T1 D and duration of the honeymoon period.

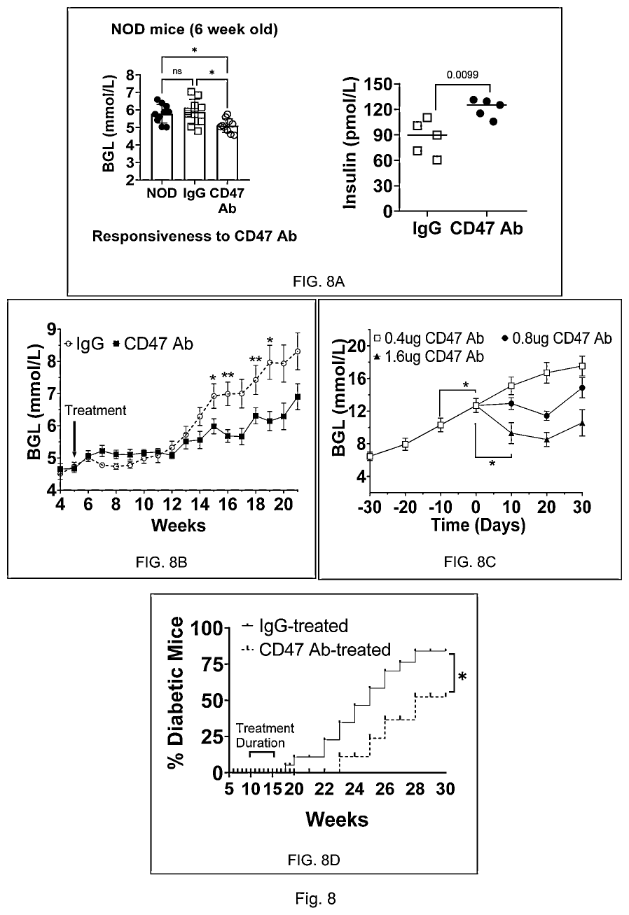

In order to test the responsiveness of NOD mice to CD47 receptor antagonism, 6-week-old NOD mice (Kew Bioservices—WEHI, Victoria 3101) (n=10/group) were injected intraperitoneally with a single dose of 0.4 μg MIAP301 (denoted by CD47Ab) for 1 h before the onset of the dark phase (1900 hours) and measured glucose levels at 0900 hours the following day. FIG. 8A, left panel, shows that mice were divided into 3 groups—NOD, IgG, CD47 Ab. The NOD group was not treated with any reagent and acted as a control to determine if IgG treatment itself would have an effect. NOD mice treated with MIAP301 displayed a significant decrease in glucose levels, which is associated with a strong trend to increased insulin levels. Specifically, FIG. 8A right panel, shows plasma insulin levels measured by ELISA after blood was withdrawn and plasma was isolated from IgG and CD47 ab-treated mice. This result indicated that β-cells in a T1 D environment are responsive to the inhibition of CD47 receptor signalling.

To determine whether MIAP301 treatment would prevent or delay the onset of diabetes in NOD mice, a cohort of NOD mice was treated daily with either MIAP301 or IgG from 5 weeks of age onwards. That is, NOD mice (n=10/group) were either treated with IgG or MIAP301 twice every week and their diabetes progression monitored over the course of 21 weeks. BLG was determined from blood extracted from a tail vein cut weekly. As expected, IgG-treated NOD mice exhibited worsening glucose control over time and eventually developed overt diabetes (FIG. 8B). In contrast, 0.4 ug CD47 Ab-treated NOD mice showed a marked resistance to the onset of hyperglycemia for an extended period (4-5 weeks) (FIG. 8B), but eventually these mice tend to progress to hyperglycemia. However, when these MIAP301-treated NOD mice were diagnosed with hyperglycemia (glucose levels >12 mM), doubling the dose of MIAP301 to 0.8 ug was able to stop the progress to diabetes for few weeks. This is shown in FIG. 8C, where mice which crossed the diabetic threshold of 12 mM were further divided into 3 groups for monitoring: 0.4 μg-treated, 0.8 μg-treated and 1.6 μg-treated. However, reversing the trend required increasing the dose to 1.6 ug i.e. 4-fold of initial dose and further delayed the onset of overt hyperglycemia (FIG. 8C).

FIG. 8D shows the difference that anti-CD47 antibody treatment makes in prevention of overt diabetes. For this, NOD mice (n=30) were treated with IgG and MIAP301 starting at week 10 of age until week 15. Only around 50% of CD47 Ab treated NOD mice succumb to diabetes after 30 weeks of age while for the IgG-treated mice, that number is higher than 75%, indicating a significant protective effect of CD47 blockade. Overall, a short-term treatment with anti-CD47 antibody provided long-term preventive benefit against the onset of diabetes. Additionally, the percent of mice that became diabetic could be substantially reduced by treatment with CD47-blocking antibody in pre-diabetic phase.

### Example 8—Effects of CD47 Deletion on Insulin Granule Docking and Secretion by β-Cells

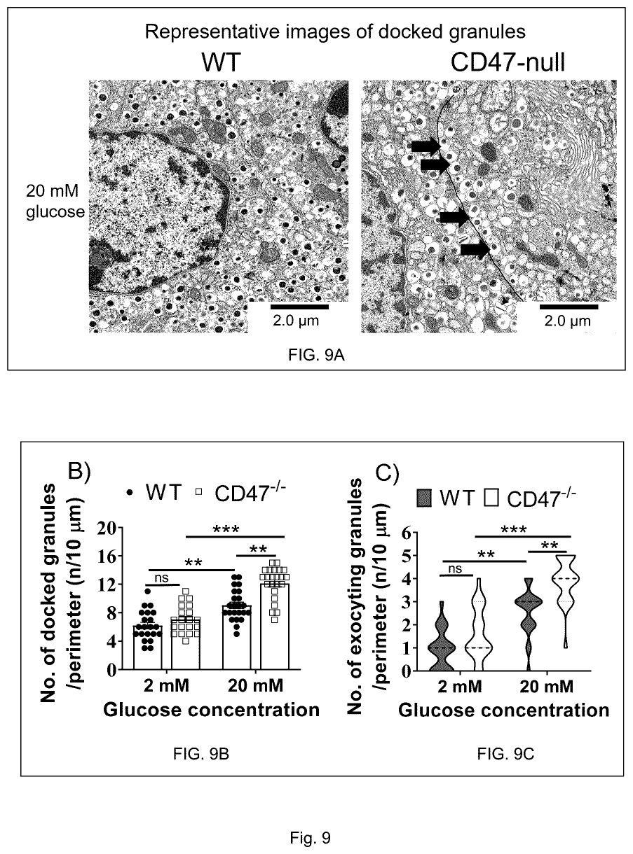

Insulin is generated by β-cells as granules and these granules dock at the β-cell membrane before release by exocytosis (see e.g. Cell. Mol. Life Sci. 78, 1957-1970 (2021)). FIG. 9 shows the effects of CD47 deletion on insulin docking and secretion by β-cells at nano-molecular resolution. WT C57/BL6 and corresponding CD47 null mutants were used (consistent with Example 4 above; Australian Bioresources, Moss Vale, NSW, Australia). For these experiments, islets were isolated as previously described with minor modifications (see Szot et al, 2007, J Vis Exp. 2007; (7):255, PMID: 18989427). In short, WT and CD47^{-/-} mice pancreases were inflated through the bile duct by inserting needle through the joint site of the hepatic duct and the cystic duct with 3 ml ice-cold liberase solution (0.5 mg/ml liberase, Merck, in Medium 199, supplemented with 1% FBS, Sigma-Aldrich). Pancreases were digested for 15-16 min at 37° C. in a water bath with gentle shaking every few minutes. Digested tissues were washed with ice-cold washing solution (Medium 199, 5 mM HEPES; 0.02% w/v BSA) and centrifuged at 800×g for 3 min at 4° C. Gradient separation and isolation of islet and pancreatic acinar tissues were performed with Ficoll 400 (Sigma-Aldrich) layered in a discontinuous gradient of 1.109, 1.096, 1.070, and 0.570 g/mL. Islets were isolated from the interfaces of both the 1.070/1.096 and 1.096/1.109 g/mL layers. islets were hand-picked with a 20 μl pipette under an inverted microscope before transferring to medium 199, supplemented with 5.5 mM glucose (D9434, Sigma-Aldrich), 10% FBS, 1× antibiotic-antimycotic solution (GIBCO, Catalog number: 15240096) at 37° C. After overnight (O/N) culture, about 50 islets from each mouse were picked and placed in a 6-well plate well containing 2 ml of KRBH solution (Hepes-buffered Krebs-Ringer, 2.56 mM CaCl₂, 5 mM KCl, 1 mM MgCl₂, 125 mM NaCl, 25 mM Hepes pH 7.4) containing 2.2 mM glucose. Islets were left for 30 min at 37° C., after which they were transferred to 20 mM glucose for GSIS experiments. After GSIS, islets from both WT and CD47^{-/-} mice were prepared for electron microscopy. In short, samples were fixed with a Karnovsky style fixative (2.5% glutaraldehyde, 4% paraformaldehyde in a 0.1 M cacodylate buffer with 5 mM CaCl₂ (pH 6.8)) for 1 hr, postfixed with 1% osmium trioxide for 30 min, then treated with 2.5% uranyl acetate for 30 min. Islets were then dehydrated using a graded series of ethanol and infiltrated with epoxy 812 resin in polyethylene capsules. Samples were sectioned on a microtome to 70-90 mm thickness and collected on 22 mesh copper grids. Sections were counterstained for 15 min using uranyl acetate, followed by lead citrate. Sections were then examined and photographed using a Hitachi HT7800 120 kV Transmission Electron Microscope (Hitachi Limited, Tokyo) according to standard protocols.

FIG. 9A compares glucose-stimulated insulin granule docking in CD47^{-/-} mice compared to WT using transmission electron microscopy (TEM). Significantly higher numbers of insulin granules were observed docked at the β-cell membrane of CD47^{-/-} mice compared to WT after glucose stimulation (FIG. 9B). In addition, more insulin granules were exocytosed from the membrane of CD47-deleted pi-cells compared to WT counterparts (FIG. 9C). This data provides unequivocal evidence which supports the benefit of modulating CD47 on insulin secretion by pi-cells.

While illustrative embodiments have been illustrated and described, including the best mode known to the inventors for carrying out the invention, those skilled in the art will recognise that the invention may be practiced with variations on the disclosed structures, materials, compositions and methods, and such variations are regarded as within the ambit of the disclosure.

The contents of all references, and published patents and patent applications cited throughout the application are hereby incorporated by reference. Full bibliographic details of references cited herein are collected at the end of the subject specification.

1. A method of preparing a transplantable pancreatic islet cell, said method comprising:
providing an isolated pancreatic islet cell; and reducing CD47 gene expression or CD47 function in said cell to produce a modified pancreatic islet cell.

2. The method of claim 1, wherein reduction of CD47 gene expression or its function increases insulin expression in and/or secretion by said modified cell when compared to insulin expression in and/or secretion by an unmodified isolated pancreatic islet cell in which CD47 expression or function is not reduced.

3. The method claim 1, wherein reduction of CD47 gene expression is mediated by gene silencing, preferably by RNA interference, an antisense oligonucleotide, or a CD47 blocking antibody or binding fragment thereof.

4. The method of claim 1, wherein said method is performed ex vivo, in vivo, or in vitro.

5. A modified pancreatic islet cell produced by the method of claim 1.

6. The modified pancreatic islet cell of claim 5, wherein which CD47 gene expression or CD47 function is reduced when compared to an unmodified pancreatic islet cell.

7. The modified pancreatic islet cell of claim 5, wherein the modified pancreatic islet cells is for use in pancreatic islet cell transplantation, wherein CD47 gene expression or CD47 function in said modified pancreatic islet cells is reduced when compared to an unmodified pancreatic islet beta cell.

8. The modified pancreatic islet beta cell of claim 6, wherein reduction of CD47 gene expression or function increases insulin expression in and/or secretion by said cell when compared to insulin expression in and/or secretion by an unmodified pancreatic islet cell in which CD47 expression is not reduced.

9. The modified pancreatic islet beta cell of claim 5, wherein reduction of CD47 gene expression or function occurs ex vivo or in vitro.

10. The modified pancreatic islet beta cell of claim 5, wherein reduction of CD47 gene expression or function is mediated by:
gene silencing, preferably by RNA interference,
an antisense oligonucleotide, or
a CD47 blocking antibody or binding fragment thereof.

11. A method of treating a disease or condition treatable by pancreatic islet cell transplantation in a subject in need, said method comprising administering to said subject an effective amount of a modified pancreatic islet beta cell in which CD47 gene expression or CD47 function is reduced when compared to an unmodified pancreatic islet beta cell.

12. The method of claim 11, wherein reduction of CD47 gene expression or function increases insulin expression in and/or secretion by said cell when stimulated with glucose when compared to insulin expression in and/or secretion by an unmodified pancreatic islet beta cell when stimulated with glucose in which CD47 expression is not reduced.

13. The method of claim 11, wherein said disease or condition is caused or associated with defective insulin production, a defective ability to utilise insulin or defective insulin signalling, preferably wherein said disease or condition is type 1 diabetes, type 2 diabetes, gestational diabetes, congenital diabetes due to genetic defects of insulin secretion, cystic fibrosis-related diabetes, steroid diabetes induced by high doses of glucocorticoids, monogenic diabetes, impaired glucose tolerance, hyperglycaemia or metabolic syndrome.

14. The method of claim 11, wherein reduction of CD47 gene expression or CD47 function, is mediated by:
gene silencing, preferably by RNA interference,
an antisense oligonucleotide, or
a CD47 blocking antibody or binding fragment thereof.

15. The method of claim 11, comprising treating a disease or condition treatable by increasing insulin expression in a subject in need, said method comprising administering to said subject an effective amount of a CD47-specific silencing molecule, preferably wherein said CD47-specific silencing molecule is a CD47-specific siRNA, blocking antibody, or morpholino.

16. The method of claim 15, wherein said method is for improving glucose clearance.

17. The method of claim 11, comprising treating diabetes in a subject, the method comprising modulating CD47 expression or function in the subject.

18. The method of claim 17, wherein treatment comprises administering to said subject an effective amount of a CD47-specific silencing molecule selected from a CD47-specific siRNA, blocking antibody or morpholino.

19.-23. (canceled)
